(19) United States
(12) Patent Application Publication
(10) Pub. No.: US 2025/0267301 A1
(43) Pub. Date: Aug. 21, 2025

(54) IMAGE PROCESSING DEVICE, IMAGE PROCESSING METHOD, AND PROGRAM

(71) Applicant: Sony Group Corporation, Tokyo (JP)

(72) Inventors: Ayumu Taguchi, Tokyo (JP); Kenji Ikeda, Kanagawa (JP); Kota Nakamura, Kanagawa (JP)

(73) Assignee: Sony Group Corporation, Tokyo (JP)

(21) Appl. No.: 18/842,198

(22) PCT Filed: Feb. 27, 2023
(86) PCT No.: PCT/JP2023/006918

§ 371 Date: Apr. 7, 2025

(30) Foreign Application Priority Data
Mar. 7, 2022 (JP) 2022-034632

(51) Int. Cl. H04N 19/44; G01N 21/64; H04N 19/86

(52) U.S. Cl. CPC H04N 19/44; G01N 21/6456; H04N 19/86

(57) ABSTRACT

To provide a technique advantageous for performing image compression processing of image data while reducing noise of the image data.

An image processing device includes a square root calculation unit that acquires first compressed image data by calculating a plurality of pixel square roots that are square roots of a plurality of original pixel values based on a plurality of pixel values included in image data, and a fraction processing unit that acquires second compressed image data by performing fraction processing of the plurality of pixel square roots.

### TECHNICAL FIELD

The present disclosure relates to an image processing device, an image processing method, and a program.

### BACKGROUND ART

An image captured and acquired by an imaging element (image sensor) includes a noise component in addition to an original image of an imaging target.

A device disclosed in Patent Document 1 performs image processing using a gradation space that is hardly affected by noise caused by an imaging element.

### CITATION LIST

### Patent Document

Patent Document 1: WO 2006/006373 A1

### SUMMARY OF THE INVENTION

### Problems to be Solved by the Invention

With the recent increase in the number of pixels and the size of image sensors, the amount of image data output from the image sensors tends to increase. For example, an image used for medical use such as a biological tissue image is required to have precise reproducibility, and thus the data amount of each image is often large, and a large number of images of the same subject may be captured and acquired.

In order to store an image having a large data amount in a storage unit as it is, a large-capacity storage unit is required, and a long time is required for data transfer. However, it takes a corresponding cost to prepare a large-capacity storage unit. In addition, data transfer processing over a long period of time increases the time required for image storage processing, and may become a bottleneck of processing of the entire system and cause other processing performed before and after the image storage processing to stagnate.

Accordingly, by performing image compression processing prior to the image storage processing, the amount of stored data is reduced. Thus, it is possible to suppress an increase in the capacity of the storage unit, shorten the time required for the image storage processing, and promote improvement in processing efficiency of the entire system and reduction in processing time.

However, by performing image compression processing (particularly lossy compression processing), partial data of an image is lost, and an original image of an imaging target may not be sufficiently reproduced in a compressed and decompressed image. In addition, such image compression processing may increase noise in the compressed and decompressed image, and image quality of the compressed and decompressed image is more likely to deteriorate.

Note that the lossless compression processing can prevent partial data loss of an image, but inherently has a small compression rate, so that the amount of image data cannot be sufficiently compressed and reduced, and an increase in capacity of the storage unit and an increase in time of the image storage processing cannot be necessarily sufficiently eliminated.

The present disclosure provides a technique advantageous for performing image compression processing of image data while reducing noise of the image data.

### Solutions to Problems

One aspect of the present disclosure relates to an image processing device including a square root calculation unit that acquires first compressed image data by calculating a plurality of pixel square roots that are square roots of a plurality of original pixel values based on a plurality of pixel values included in image data, and a fraction processing unit that acquires second compressed image data by performing fraction processing of the plurality of pixel square roots.

The plurality of original pixel values may be derived by dividing a plurality of pixel values included in the image data by a reference pixel value that is an output value corresponding to one electron in photoelectric conversion processing of the imaging element that has acquired the image data.

The fraction processing unit may acquire the second compressed image data by rounding a numerical value after a decimal point of each of the plurality of pixel square roots on the basis of rounding off.

The fraction processing unit may obtain an approximation original pixel value that is n\hat{ }2 (n is a natural number) having a smallest difference with respect to an original pixel value corresponding to each of the pixel square roots for each of the plurality of pixel square roots, and employ n of the approximation original pixel value as a pixel value of the second compressed image data.

The image processing device may include a word length adjustment unit that acquires third compressed image data by reducing a word length of the second compressed image data according to sizes of a plurality of pixel values included in the second compressed image data.

The word length adjustment unit may acquire the third compressed image data by reducing the word length of the second compressed image data according to a number of significant digits of a maximum value among the plurality of pixel values included in the second compressed image data.

The second compressed image data may have a word length equal to or more than 16 bits, and the third compressed image data may have a word length equal to or less than 8 bits.

The image processing device may include a compression processing unit that acquires fourth compressed image data by performing compression processing on compressed image data based on the second compressed image data.

The image processing device may include a word length adjustment unit that acquires third compressed image data by reducing a word length of the second compressed image data according to sizes of a plurality of pixel values included in the second compressed image data, in which the compression processing unit may acquire fourth compressed image data by performing compression processing on the third compressed image data.

The image processing device may include a storage processing unit that causes the compressed image data based on the second compressed image data to be stored in the storage unit.

The image processing device may include a decoding unit that performs processing of squaring a plurality of pixel values included in the compressed image data based on the second compressed image data.

The image processing device may include a word length restoration unit that increases a word length of the compressed image data based on the second compressed image data, in which the decoding unit may perform processing of squaring a plurality of pixel values included in the compressed image data based on the second compressed image data after the word length of the compressed image data based on the second compressed image data is increased by the word length restoration unit.

The image processing device may include a compression processing unit that acquires fourth compressed image data by performing compression processing of the compressed image data based on the second compressed image data, and a decompression processing unit that performs decompression processing on the compressed image data based on the second compressed image data, in which the compressed image data to be decompressed may be based on the fourth compressed image data, and the decompression processing may be processing corresponding to the compression processing performed by the compression processing unit.

The plurality of original pixel values may be derived by dividing a plurality of pixel values included in the image data by a reference pixel value that is an output value corresponding to one electron in photoelectric conversion processing of the imaging element that has acquired the image data, and the decoding unit may acquire a plurality of decompressed pixel values by multiplying a plurality of squared pixel values obtained by squaring a plurality of pixel values included in the compressed image data based on the second compressed image data by the reference pixel value.

The image data may be biological tissue image data.

The biological tissue image data may be fluorescent antibody image data.

Another aspect of the present disclosure relates to an image processing method including a step of acquiring first compressed image data by calculating a plurality of pixel square roots that are square roots of a plurality of original pixel values based on a plurality of pixel values included in image data, and a step of acquiring second compressed image data by performing fraction processing of the plurality of pixel square roots.

Another aspect of the present disclosure relates to a program for causing a computer to execute a procedure of acquiring first compressed image data by calculating a plurality of pixel square roots that are square roots of a plurality of original pixel values based on a plurality of pixel values included in image data, and a procedure of acquiring second compressed image data by performing fraction processing of the plurality of pixel square roots.

### BRIEF DESCRIPTION OF DRAWINGS

FIG. 1 is a conceptual diagram for describing photoelectric conversion in an imaging element (particularly, a pixel).

FIG. 2 is a conceptual diagram of pixel values which are 16 bit pixel data.

FIG. 3 is a conceptual diagram of pixel values which are 16 bit pixel data.

FIG. 4 is a conceptual diagram of pixel values which are 16 bit pixel data.

FIG. 5 is a conceptual diagram of pixel values which are 16 bit pixel data.

FIG. 6 is a conceptual diagram of pixel values which are 16 bit pixel data.

FIG. 7 is a block diagram illustrating a configuration example of an imaging processing system.

FIG. 8 is a block diagram illustrating a functional configuration example of the image processing device, and particularly illustrates a functional configuration related to image compression processing.

FIG. 9 is a block diagram illustrating a functional configuration example of the image processing device, and particularly illustrates a functional configuration related to image decompression processing.

FIG. 10 is a flowchart illustrating an example of the image compression processing.

FIG. 11 is a conceptual diagram of pixel data for describing calculation of a square root of pixel data.

FIG. 12 is a diagram for describing a fraction processing example.

FIG. 13 is a flowchart illustrating an example of image decompression processing.

FIG. 14 is a block diagram illustrating a configuration example of a microscope system.

FIG. 15 is a diagram illustrating an example of an imaging device.

FIG. 16 is a diagram illustrating an example of a mobile device.

FIG. 17 is a diagram illustrating an example of a client server system.

### MODE FOR CARRYING OUT THE INVENTION

Typical embodiments of the present disclosure will be described with reference to the drawings.

Hereinafter, a case where the image data to be processed is biological tissue image data (for example, fluorescent antibody image data) will be mainly exemplified. However, the image data to which the technology described below can be applied is not limited. The technology described below is also applicable to image data used in any application other than medical care, and is also applicable to, for example, a normal snap photograph.

In the following description, the image data is also simply referred to as an “image”, and collectively includes a large number of pixel values (pixel data).

In a biological tissue image, due to its specific application, appropriate retention of original image quality may be required not only in a bright image region but also in a dark image region.

For example, a researcher or a doctor visually recognizes not only a fine tissue structure of a bright image region but also a fine tissue structure of a dark image region, and observes texture such as roughness that an ordinary person would overlook, thereby determining the presence or absence of cell abnormality such as cancer.

In a case where a biological tissue image to be observed includes a plurality of frequency components (a plurality of frequency patterns), since a certain frequency component lowers sensitivity of other frequency components, it is difficult for an ordinary people to recognize a fine tissue structure in such a biological tissue image. On the other hand, a researcher or a doctor who is familiar with the fact that sensitivity of each frequency component changes by performing observation while changing the distance and angle of the eye with respect to such an image can appropriately recognize the fine tissue structure in the biological tissue image by distinguishing subtlety of each frequency component.

In addition, for other purposes, it may be required to perform observation particularly focusing on a dark image region in a biological tissue image including both a bright image region and a dark image region.

Thus, regarding the biological tissue image, it is required to ensure a wider dynamic range and maintain a fine texture as compared with a normal snap photograph or the like.

For example, in biological tissue observation by fluorescence microscopy, the type and amount of protein fluorescently expressed in a biological tissue range to be observed greatly vary, and the light emission efficiency and the light emission amount (fluorescence amount) of the fluorescent antibody may greatly vary between observation sites. The fluorescent antibody image obtained in such a case may include both a bright image region and a dark image region.

However, unlike a normal snap photograph, the fluorescent antibody image is imaged in a state in which the irradiation light amount of excitation light is suppressed in order to suppress deterioration of the tissue due to the excitation light that excites he fluorescent antibody molecules. Thus, the amount of fluorescence signal contained in the fluorescent antibody image is originally small, and the fluorescent antibody image tends to be dark as a whole.

As described above, in a biological tissue image such as a fluorescent antibody image including a dark image region partially or entirely in this manner, the influence of a noise component such as shot noise becomes relatively large, and the noise component is often included in the biological tissue image at a non-negligible level.

Thus, it is preferable that image processing for reducing the noise component is performed on the biological tissue image so that the original image of the subject can be appropriately observed in the biological tissue image.

As such an image processing method, there is a method of acquiring a plurality of fluorescent antibody images by performing imaging a plurality of times, and acquiring a fluorescent antibody image in which a noise component is reduced on the basis of averaging of the plurality of fluorescent antibody images. However, in this method, irradiation of a tissue with excitation light is performed a plurality of times, and deterioration of the tissue due to such irradiation of the excitation light a plurality of times is concerned. Thus, it is often difficult to employ this method.

In the case of acquiring a biological tissue image, the entire slide including a biological tissue to be observed is imaged using light of various wavelengths (for example, 100 or more wavelengths), and such imaging may be performed for each of a plurality of slides.

That is, in a case where a biological tissue image is acquired, there is a case where only an image in a single field of view of a microscope is captured and stored, but there is also a case where an image of the entire slide is acquired and stored by performing imaging while scanning the entire slide. In addition, in a case of acquiring a fluorescence microscope image, imaging may be performed using excitation light of various wavelengths.

The data amount of the biological tissue image obtained by such imaging is enormous.

Storage of such enormous image data causes an increase in cost of a storage (storage unit). In addition, as the data amount of the image to be saved increases, the time required for data transfer to the storage increases. The increase in the time for data transfer may become a bottleneck that hinders improvement in performance of the entire system.

For example, in a case where an image of “30,000 px×30,000 px (16 bits)” is stored at a wavelength of 335 channel CH, an image data amount is approximately 603 GB per slide. In a case where the image data is saved in a hard disk drive (HDD) at a transfer speed of 255 MB/s, for example, it takes about 39.41 minutes from the start to the end of saving the image data.

In this case, the time required to transfer and store the image greatly exceeds the time required to capture and acquire the image, which is a factor that greatly delays the processing speed of the entire system.

In a case where a SATA SSD (transfer speed: 750 MB/s) or an M.2 SSD (transfer speed: 2800 MB/s) having a transfer speed higher than that of the HDD is used as the storage, the time from the start to the end of the saving of the image data is about 13.40 minutes and 3.59 minutes, respectively. By using the storage with a high transfer speed in this manner, the time required to store the image data is shortened, and the system performance is greatly improved.

However, a storage having such excellent data transfer speed performance is quite expensive, and may be difficult to employ in terms of cost.

In order to suppress the increase in storage cost and prolongation of the data transfer time as described above, it is effective to reduce the data amount of the biological tissue image to be stored by image compression processing prior to storing in the storage.

However, when general-purpose image compression processing is applied to a biological tissue image, a dynamic range (for example, texture in a dark image region) is sacrificed.

For example, in JPEG using DCT compression, it is possible to acquire compressed image data with a high compression rate by setting the quantization level of the DCT compression to a level sufficiently higher than shot noise in consideration of the brightness of the entire image. However, since such compression processing is processing for reducing the amount of image data by sacrificing the dynamic range, texture in a dark image region is lost.

In a normal snap photograph, the influence of density of a dark image region adjacent to a bright image region on the texture of the entire image is small, and thus, even if the texture in the dark image region is lost by the compression processing, there is almost no practical disadvantage in observation in many cases.

On the other hand, general-purpose compression processing such as DCT compression sacrificing a dynamic range is not suitable for a biological tissue image for which precise texture maintenance is required even in a dark image region.

In addition, since a biological tissue image is required to have precise image reproducibility, the biological tissue image tends to have a large bit depth, and for example, it is not uncommon that the biological tissue image has a word length equal to or more than 16 bits.

On the other hand, many general-purpose image compression libraries (image encoders) are applicable to images having a word length equal to or less than 8 bits, but the general-purpose image compression libraries applicable to images having a word length equal to or more than 16 bits are very limited.

Thus, there is no general-purpose image compression library capable of compressing a high-bit image with high compression efficiency while ensuring a wide dynamic range and suppressing an increase in noise.

Note that there is an image that originally has an enormous data size other than the biological tissue image, and there is a technical problem similar to the above-described biological tissue image regarding handling of such an image that has an enormous data size.

Hereinafter, an image processing technique advantageous for reducing noise components such as shot noise while compressing image data will be exemplarily described.

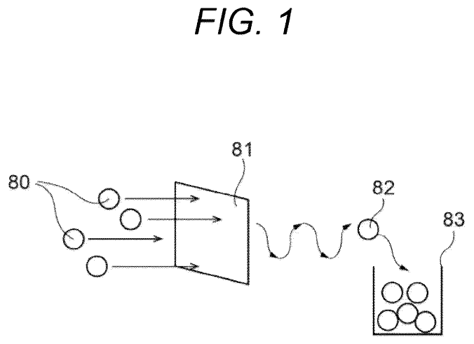

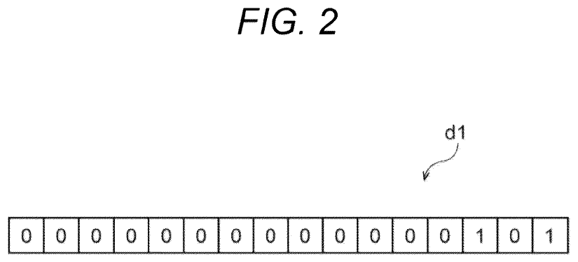

FIG. 1 is a conceptual diagram for describing photoelectric conversion in an imaging element (in particular, a pixel 81). FIG. 2 is a conceptual diagram of a pixel value d1 which is 16 bit pixel data.

As illustrated in FIG. 1, the pixel 81 of the imaging element outputs electrons 82 corresponding to received photons 80, and the electrons 82 output from the pixel 81 are accumulated in an electron storage unit 83.

Then, by performing digital conversion (A/D conversion) of the voltage (the number of electrons 82) accumulated in the electron storage unit 83, a pixel value d1 (pixel data) of digital data (16 bit digital data (binary data) in the example illustrated in FIG. 2) is output.

The pixel data (image data) output from the imaging element in this manner includes shot noise as a main noise component.

The shot noise is a kind of circuit noise inevitably generated according to photoelectric conversion performed in each pixel 81, and is based on an inherent error associated with measurement of particles such as photons and electrons.

The magnitude of the shot noise is proportional to the square root of intensity of observation light or an average value of current, and as the observation light intensity and the current value are smaller, the proportion of the absolute value of the shot noise in the pixel value d1 output from each pixel 81 tends to increase.

Thus, it is possible to reduce the ratio of shot noise in captured image data by increasing the illumination light amount (for example, an output of an excitation light source that excites fluorescent molecules) at the time of imaging.

However, in practice, considering the damage that can be caused to the biological sample and the fluorescent antibody reagent by the light at the time of image capturing, it is often impossible to perform long-time exposure and multiple times exposure on the biological tissue to be captured.

The shot noise increases information entropy (that is, unpredictability), causes a decrease in efficiency of image compression in both lossless compression processing and lossy compression processing, and does not provide useful information to the observer.

On the other hand, it is inherently impossible to make the shot noise smaller than the square root of the signal light amount (that is, the number of electrons generated in the semiconductor of the image sensor) due to quantum mechanical constraints. That is, the shot noise is a noise component generated on the basis of the physical essence, and the shot noise cannot be made smaller than the square root of the signal light amount except in a very special situation.

Since the shot noise is equal to or proportional to the square root of an expected value of light intensity detected at each pixel, the noise level of the shot noise may vary from pixel to pixel. Thus, it is not possible to uniformly reduce the shot noise by uniformly setting the noise level of the shot noise over the entire image (that is, all pixels).

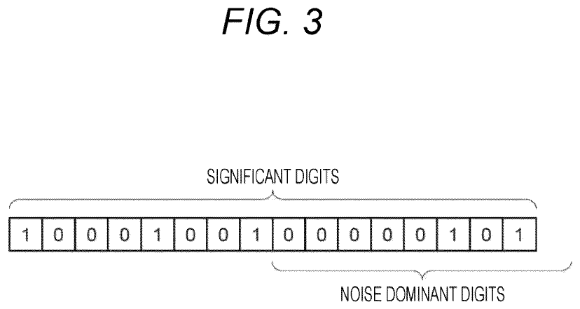

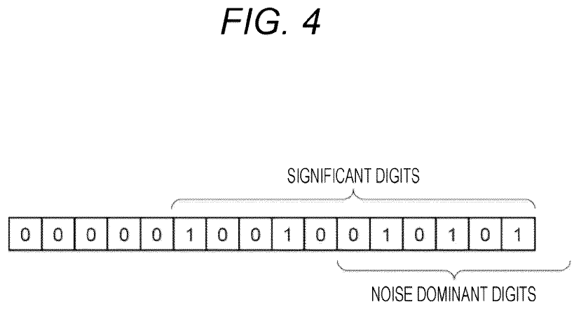

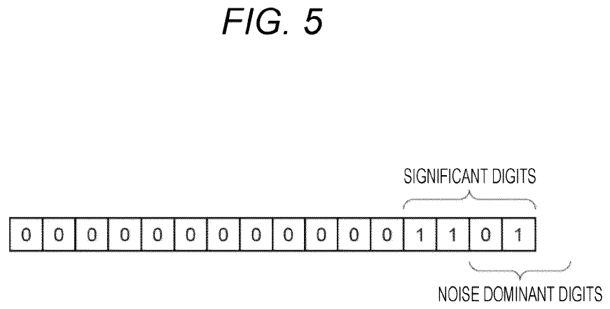

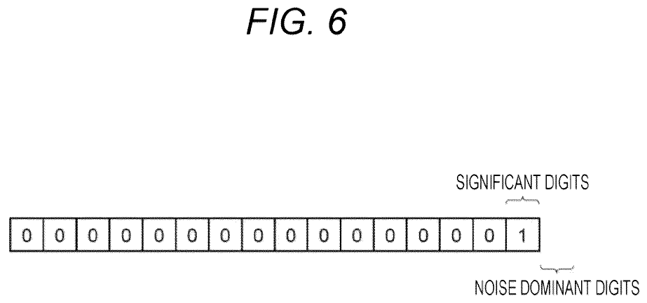

FIGS. 3 to 6 are conceptual diagrams of a pixel value d1 which is 16 bit pixel data. FIGS. 3 to 6 illustrate pixel values d1 having different numbers of significant digits.

The standard deviation of the shot noise is equal to or proportional to the square root (=d1\hat{ }(1/2)) of the pixel value d1 (that is, a pixel signal value (detected light amount value)).

The number of digits of the shot noise is expressed by “Log [d1\hat{ }(1/2)]=1/2×Log [d1]”.

As is clear from this expression, the shot noise occupies approximately the lower half digit of the significant digit of the pixel value d1. That is, the digits of the lower half of the significant digits of the pixel value d1 represented in binary notation are noise dominant digits (that is, a shot noise floor) dominated by the shot noise (see FIGS. 3 to 6), and barely include intrinsic information regarding the subject image.

The image compression technology described below is based on this knowledge, and focuses on the fact that the numerical value of the lower order digit in which the shot noise is dominant among the significant digits of each pixel value d1 (light intensity signal of each pixel) does not have much meaning as the data representing the subject image.

That is, primary image compression is performed by reducing the number of digits of the image data to be stored according to the number of digits of the shot noise floor. Thus, the data amount of the image data can be compressed to about half, and the shot noise that is not an intrinsic component of the image data can be reduced to lower the information entropy.

As a result, the amount of image data actually stored in the storage unit is significantly reduced, and a storage cost can be significantly reduced and a data transfer time can be significantly shortened (furthermore, a storage processing time can be significantly shortened).

In addition, by the image compression processing, the shot noise floor is removed from the image data, and only a portion corresponding to the shot noise component is reduced, so that deterioration in image quality is quite small. Furthermore, in a case where additional image compression processing is performed on the image data in which the shot noise is reduced by the image compression processing, improvement in efficiency of such additional image compression processing can also be expected.

Hereinafter, a specific device configuration example and a specific processing configuration example will be described.

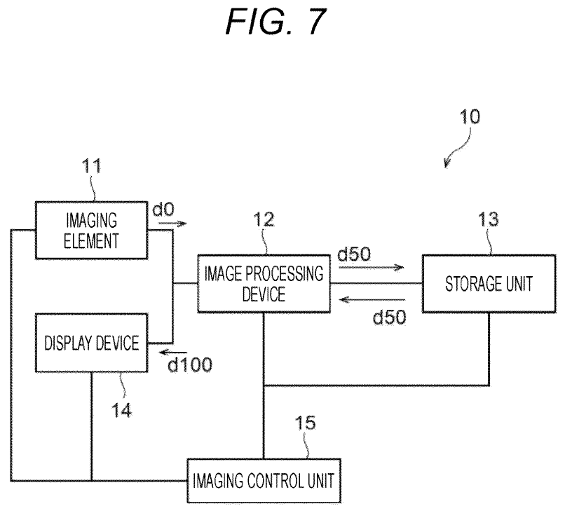

FIG. 7 is a block diagram illustrating a configuration example of the imaging processing system 10.

An imaging processing system 10 illustrated in FIG. 7 includes an imaging element 11, an image processing device 12, a storage unit 13, a display device 14, and an imaging processing control unit 15. The imaging processing system 10 may be configured by a single device or may be configured by combining a plurality of devices.

The imaging element 11, the image processing device 12, the storage unit 13, and the display device 14 are driven under the control of the imaging processing control unit 15.

The imaging element 11 receives imaging light from a subject and outputs image data do including a subject image.

The imaging element 11 can have any configuration, and is typically constituted by a CCD image sensor or a CMOS image sensor.

The image data do acquired by the imaging element 11 is not limited, and may be biological tissue image data (for example, fluorescent antibody image data) or image data obtained by imaging any other object.

The image data do output from the imaging element 11 is input to the image processing device 12, and undergoes various types of image processing in the image processing device 12.

The image processing performed by the image processing device 12 is not limited, but the image processing device 12 of the present embodiment performs at least image compression processing and image decompression processing.

That is, the image processing device 12 generates compressed image data d50 by performing the image compression processing on the image data do, and outputs the compressed image data d50 to the storage unit 13. The storage unit 13 stores the compressed image data d50 output from the image processing device 12.

In addition, the image processing device 12 generates decompressed image data d100 by performing image decompression processing on the compressed image data d50 read from the storage unit 13, and outputs the decompressed image data d100 to the display device 14. The display device 14 displays the decompressed image data d100 output from the image processing device 12.

The decompressed image data d100 output from the image processing device 12 may be transmitted to any other device in addition to the display device 14 or instead of the display device 14.

In the example illustrated in FIG. 7, the image compression processing and the image decompression processing are performed by the common image processing device 12, but the image compression processing and the image decompression processing may be performed by separate image processing devices 12.

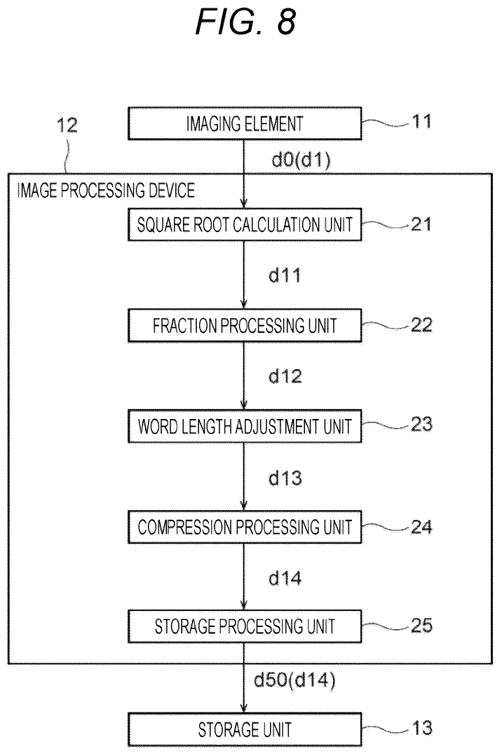

FIG. 8 is a block diagram illustrating a functional configuration example of the image processing device 12, and particularly illustrates a functional configuration related to the image compression processing.

The image processing device 12 illustrated in FIG. 8 includes a square root calculation unit 21, a fraction processing unit 22, a word length adjustment unit 23, a compression processing unit 24, and a storage processing unit 25.

The square root calculation unit 21 acquires first compressed image data d11 by calculating a plurality of pixel square roots that are respective square roots of a plurality of original pixel values based on a plurality of pixel values d1 included in the image data do input from the imaging element 11.

Here, the plurality of original pixel values is derived by dividing each of the plurality of pixel values d1 included in the image data do by a reference pixel value that is an output value corresponding to one electron in the photoelectric conversion processing of the imaging element 11 that has acquired the image data do.

Alternatively, the plurality of original pixel values may be the plurality of pixel values d1 itself included in the image data do. In this case, the square root calculation unit 21 acquires the first compressed image data d11 by dividing the calculated square root of each pixel value d1 by the square root of the reference pixel value.

The reference pixel value used here is represented by the absolute value of “output value k [LSB] per electron (LSB: Least Significant Bit)” in each pixel 81 (see FIG. 1) of the imaging element 11. That is, 1 [LSB] of the output value (pixel value) of each pixel 81 of the imaging element 11 does not necessarily correspond to “one electron”, and a value amplified on the basis of a certain coefficient k is often output from each pixel 81. Thus, each pixel 81 of the imaging element 11 outputs a pixel value d1 based on the relationship of “1 electron=k [LSB]”.

Therefore, as described above, by performing the square root calculation after dividing each pixel value d1 by the reference pixel value k or by performing division by using the square root of the reference pixel value k after calculating the square root of each pixel value d1, the pixel value d1 is adjusted so that 1 [LSB] corresponds to “one electron”. As a result, the shot noise and quantization noise can be made approximately equal, and the number of significant digits of the pixel intensity information can be reduced while substantial missing of information is suppressed.

The fraction processing unit 22 performs fraction processing on a plurality of pixel square roots included in the first compressed image data d11 to acquire integer-converted second compressed image data d12. For example, the fraction processing unit 22 can acquire the second compressed image data d12 by rounding off the numerical value after the decimal point (that is, the fractional part) of each of the plurality of pixel square roots on the basis of rounding off.

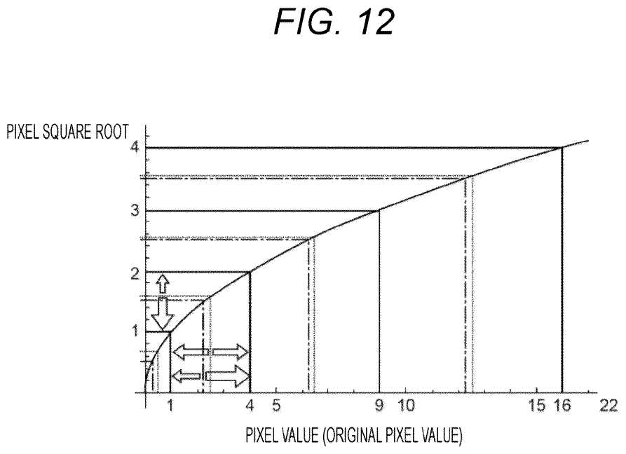

However, the fraction processing unit 22 can also perform the fraction processing in a manner other than rounding off (see FIG. 12 described later).

The word length adjustment unit 23 acquires third compressed image data d13 by reducing the word length of the second compressed image data d12 according to the magnitudes of the plurality of pixel values included in the second compressed image data d12.

For example, the word length adjustment unit 23 can acquire the third compressed image data d13 by reducing the word length of the second compressed image data d12 according to the number of significant digits of the maximum value among the plurality of pixel values included in the second compressed image data d12. Thus, it is also possible to derive the third compressed image data d13 having a word length equal to or less than 8 bits from the second compressed image data d12 having a word length equal to or more than 16 bits.

The compression processing unit 24 performs the compression processing on the third compressed image data d13 to acquire fourth compressed image data d14. The compression processing unit 24 can perform the compression processing in any manner, and may perform the compression processing using the general-purpose image compression library.

The storage processing unit 25 causes the compressed image data d50 based on the fourth compressed image data d14 to be stored in the storage unit 13. The compressed image data d50 may be the fourth compressed image data d14 itself, or may be image data derived by the storage processing unit 25 performing further processing on the fourth compressed image data d14.

Note that the third compressed image data d13 and the fourth compressed image data d14, which are processing target data in the compression processing unit 24 and the storage processing unit 25 described above, both correspond to “compressed image data based on the second compressed image data d12”.

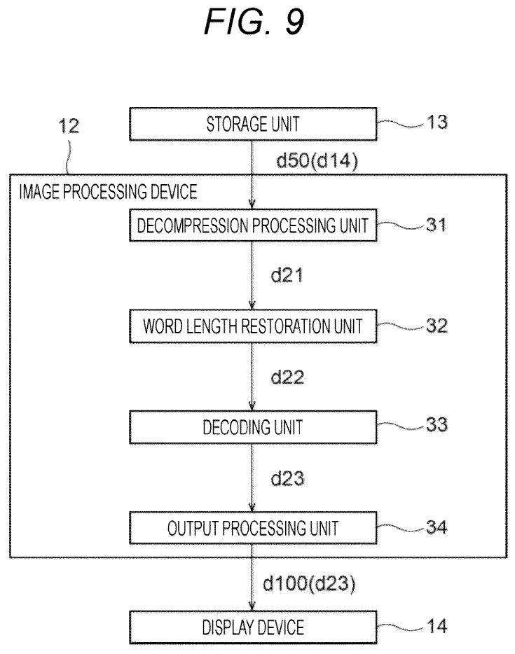

FIG. 9 is a block diagram illustrating a functional configuration example of the image processing device 12, and particularly illustrates a functional configuration related to image decompression processing.

In a case where the image compression processing and the image decompression processing are performed by the common image processing device 12, the image processing device 12 has a functional configuration related to the image decompression processing illustrated in FIG. 9 in addition to the functional configuration related to the image compression processing illustrated in FIG. 8. In a case where the image compression processing and the image decompression processing are performed by the separate image processing devices 12, one image processing device 12 has a functional configuration related to the image compression processing illustrated in FIG. 8, and the other image processing device 12 has a functional configuration related to the image decompression processing illustrated in FIG. 9.

The image processing device 12 illustrated in FIG. 9 includes a decompression processing unit 31, a word length restoration unit 32, a decoding unit 33, and an output processing unit 34.

The decompression processing unit 31 performs decompression processing on the compressed image data d50 (that is, the fourth compressed image data d14) read from the storage unit 13 to acquire first decompressed image data d21. The decompression processing performed by the decompression processing unit 31 in this manner is processing corresponding to the compression processing performed by the above-described compression processing unit 24 (see FIG. 8).

The word length restoration unit 32 increases the word length of the first decompressed image data d21 to acquire second decompressed image data d22. That is, the word length restoration unit 32 acquires the second decompressed image data d21 by increasing the word length of the first decompressed image data d22 by the same word length as the word length reduced by the word length adjustment unit 23 (see FIG. 8) in the above-described image compression processing.

The decompression processing performed by the word length restoration unit 32 in this manner is processing corresponding to the compression processing performed by the word length adjustment unit 23 described above. The word length of the second decompressed image data d22 output from the word length restoration unit 32 is the same word length as the word length of the image data do before the above-described compression processing (FIG. 8) is performed.

The decoding unit 33 performs processing of squaring a plurality of pixel values included in the second decompressed image data d22. That is, the decoding unit 33 performs processing of squaring a plurality of pixel values included in the second decompressed image data d22 after the word length of the first decompressed image data d21 is increased and the second decompressed image data d22 is acquired by the word length restoration unit 32.

Then, the decoding unit 33 obtains a plurality of decompressed pixel values by multiplying a plurality of squared pixel values obtained by squaring a plurality of pixel values included in the second decompressed image data d22 by the reference pixel value. Alternatively, the decoding unit 33 may acquire a plurality of decompressed pixel values by multiplying a plurality of pixel values included in the second decompressed image data d22 by a square root of the reference pixel value and then squaring the multiplied pixel values. The reference pixel value used in the decoding unit 33 in this manner is also used when the above-described square root calculation unit 21 (see FIG. 9) acquires a plurality of original pixel values from the pixel value d1 included in the image data do.

The decompression processing performed by the decoding unit 33 as described above is processing corresponding to square root calculation processing performed by the square root calculation unit 21 described above.

The decoding unit 33 outputs third decompressed image data d23 including a plurality of decompressed pixel values to the output processing unit 34. The output processing unit 34 outputs the third decompressed image data d23 to the display device 14 as the decompressed image data d100. The display device 14 displays the decompressed image data d100 (the third decompressed image data d23 in the present embodiment) input from the output processing unit 34.

All of the fourth compressed image data d14, the first decompressed image data d21, and the second decompressed image data d22, which are processing target data in the decompression processing unit 31, the word length restoration unit 32, and the decoding unit 33 described above, correspond to compressed image data based on the second compressed image data d12.

Next, an example of an image processing method (that is, image compression processing and image decompression processing) performed by the above-described image processing device 12 will be described.

### [Image Compression Processing]

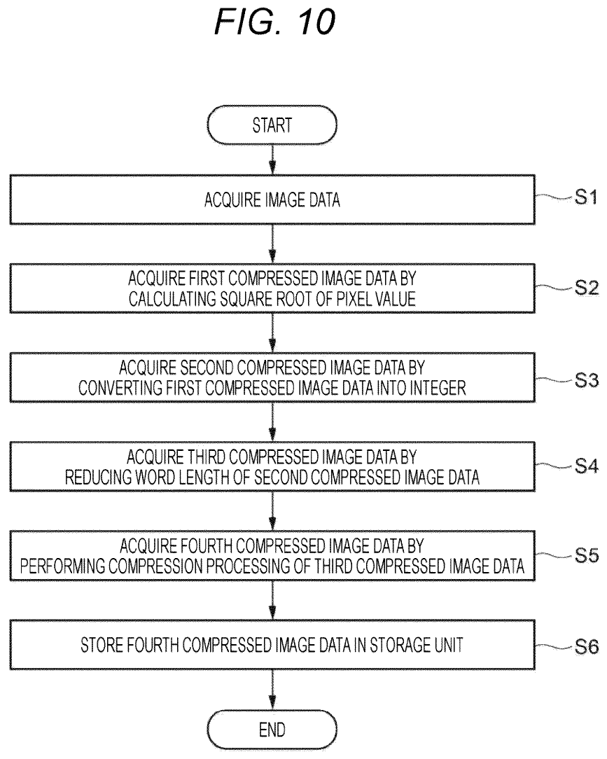

FIG. 10 is a flowchart illustrating an example of the image compression processing.

First, image data do to be processed is input to the image processing device 12 (see FIG. 8) (S1 in FIG. 10).

Then, the square root calculation unit 21 (see FIG. 8) of the image processing device 12 calculates a square root of each pixel value d1 (a plurality of pixel square roots) of the image data do and acquires the first compressed image data d11 (S2 in FIG. 10).

In the present embodiment, the biological tissue image is used as the image data do, but the image that can be used as the image data do is not limited. The biological tissue image available as the image data do is not limited, and for example, a bright field microscope image, a dark field microscope image, or a fluorescence microscope image may be input to the image processing device 12 as the image data do.

In a case where the microscopic image is used as the image data do, the subject included in the image data do is not limited to the target in the visual field, and may be the entire slide by performing imaging while scanning the entire slide once or a plurality of times. Furthermore, in a case where the fluorescence microscope image is used as the image data do, imaging is performed while the wavelength of the excitation light with which the imaging target is irradiated is changed, and a plurality of pieces of image data do having different wavelengths of the excitation light may be acquired and input to the image processing device 12. In addition, the slide of the imaging target may be automatically replaced, the imaging element 11 may automatically image the slide of the target, and the image data do of each slide may be automatically input from the imaging element 11 to the image processing device 12.

In this processing step S2, calculating the square root of each original pixel value of the image data do corresponds to making a quantization level (that is, a value corresponding to one bit) for each original pixel value equivalent to shot noise.

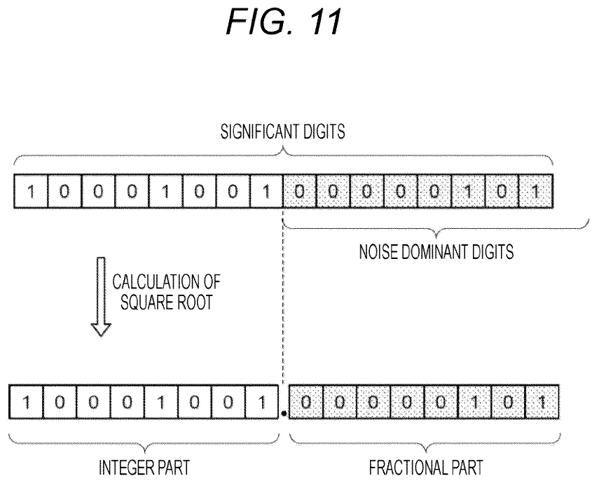

FIG. 11 is a conceptual diagram of pixel data for describing calculation of a square root of pixel data.

The processing of calculating the square root of the pixel data is processing of halving the number of significant digits of the integer part of the pixel data.

For example, as illustrated in FIG. 11, in the square root of the original pixel data having the number of significant digits of 16 bits, the value of the upper half (upper 8 bits) of the original significant digits corresponds to the integer part, and the value of the lower half (lower 8 bits) of the original significant digits corresponds to the fractional part.

On the other hand, as described above, the shot noise included in each pixel data corresponds to the lower half digits (shot noise floor) of the significant digits of the pixel data (see FIGS. 3 to 6), and the shot noise floor does not include or hardly includes the data of the original subject image.

Therefore, in this processing step S2, calculating the square root of each original pixel value corresponds to calculating a pixel value in which most of the shot noise is represented by a fractional part and most of the data of the original subject image is represented by an integer part.

Note that, in general, it is considered that quantization noise tends to increase as the quantization level increases. However, in the present embodiment, since the square root of each original pixel value is calculated in order to acquire the first compressed image data d11 as described above, the quantization level is equivalent to the shot noise. Thus, in this processing step S2 of acquiring the first compressed image data d11, the influence of the quantization noise on each pixel value is very limited.

Since the level of shot noise is equal to or proportional to the square root of each pixel value d1 (signal value of each pixel), for example, in a dark image region in the image data do, the intensity value of the shot noise is absolutely low, and in a bright image region, the intensity value of the shot noise is absolutely high.

Thus, when quantization is performed at the same level for the entire region of the image data do, only the noise signal component can be reduced in the bright image region, but the subject image component may be reduced together with the noise signal component in the dark image region.

On the other hand, by calculating the square root of each original pixel value of the image data do as in the present embodiment, the substantial quantization level for each pixel is changed according to the magnitude of the original pixel value (pixel value d1). Therefore, in both the bright image region and the dark image region, it is possible to effectively prevent a signal component meaningful as an image from being cut off by quantization.

Then, the fraction processing unit 22 (see FIG. 8) of the image processing device 12 performs the fraction processing on each pixel value (that is, each pixel square root) of the first compressed image data d11, and the fractional part of each pixel square root is rounded (S3 in FIG. 10). The second compressed image data d12 is acquired by converting each pixel value into an integer in this manner, and each of the plurality of pixel values included in the second compressed image data d12 becomes an integer value.

For example, the fraction processing unit 22 may convert each pixel value into an integer by rounding the numerical value at the first decimal place of each pixel square root on the basis of “rounding off”. Alternatively, the fraction processing unit 22 may convert each pixel value into an integer on the basis of any other method.

Specifically, fraction processing based on rounding off can be performed on the basis of the following Expression (1).

$$A = Integerpart [ Sqrt [ x ] + 0.5 ] \qquad Expression ( 1 )$$

In the above Expression (1), “A” represents a value (that is, an integer value) derived by fraction processing, and “x” represents each pixel value (each pixel square root) of the first compressed image data d11 that is the original value. “Sqrt [x]” represents a square root calculation operator of x, and “IntegerPart [Q]” represents an operator for extracting an integer part of Q.

Fraction processing based on rounding off represented by the above Expression (1) has an advantage that calculation is simple, but is not a method for minimizing quantization noise.

FIG. 12 is a diagram for describing a fraction processing example.

In FIG. 12, the horizontal axis represents a pixel value (original pixel value), and the vertical axis represents a pixel square root that is the square root of the pixel value. A solid curve illustrated in FIG. 12 indicates a correspondence between the pixel value and a pixel square root.

A solid line extending between the curve illustrated in FIG. 12 and the vertical axis and the horizontal axis in FIG. 12 indicates a case where both the corresponding pixel value and the pixel square root are integer values.

A straight line of a one-dot chain line extending between the curve illustrated in FIG. 12 and each of the vertical axis and the horizontal axis in FIG. 12 corresponds to a boundary in a case where fraction processing of a pixel square root is performed on the basis of rounding off.

A dotted straight line extending between the curve illustrated in FIG. 12 and each of the vertical axis and the horizontal axis in FIG. 12 corresponds to a boundary in a case where fraction processing of a pixel square root is performed on the basis of a rounding reference value that is the square root of a center value between n\hat{ }2 (n is a natural number).

A case will be considered where, after square root compression is tentatively applied to a random variable continuously distributed with equal probability in the range from 0 to a measurement maximum value, a variance of an original random variable is minimized. In this case, an expected value of the quantization level can be minimized by performing fraction processing (that is, rounding down processing and rounding up processing) using the square root of a center value (middle value) between “n\hat{ }2 (n is a natural number)” as a boundary value (incorporation boundary value).

Here, the “center value between n\hat{ }2 (n is a natural number)” is represented by, for example, (n²+(n+1)²)/2. Therefore, the “square root of the center value between n\hat{ }2 (n is a natural number)” is represented by, for example, {(n²+(n+1)²)/2}\hat{ }^{(1/2)}. For example, a pixel square root larger than “n (n is a natural number)” and smaller than “n+1” may be rounded to “n+1” by fraction processing in a case where the square root is equal to or larger than a rounding reference value represented by {(n²+(n+1)²)/2} \hat{ }^{(1/2)}. On the other hand, in a case where the pixel square root is smaller than the rounding reference value, the square root of the pixel may be rounded to “n” by fraction processing.

It can be said that the case of performing the fraction processing of the pixel square root using the square root of the center value between n\hat{ }2 (n is a natural number) as a rounding reference value is more suitable for the actual situation than the case of applying rounding off to the pixel square root for converting the pixel square root into an integer. That is, for each square root of pixels, an approximation original pixel value that is n\hat{ }2 (n is a natural number) having the smallest difference with respect to the pixel value (original pixel value) corresponding to each pixel square root may be obtained, and n for the approximation original pixel value may be employed as the pixel value of the second compressed image data d12.

In a case where rounding off is applied to the pixel square root, the integer value after the fraction processing changes above and below a one-dot chain line (incorporation boundary) extending in a horizontal direction from the vertical axis in FIG. 12. On the other hand, in a case where the fraction processing in which the square root of the center value between n\hat{ }2 (n is a natural number) is employed as the rounding reference value is applied to the pixel square root, the integer value after the fraction processing changes above and below the dotted line (incorporated boundary) extending in the horizontal direction from the vertical axis in FIG. 12. Therefore, the pixel square root located between the one-dot chain line and the dotted line extending in the horizontal direction from the vertical axis in FIG. 12 is converted into an integer value not conforming to the actual situation when rounding off is used for the fraction processing, which leads to an increase in quantization noise.

On the other hand, as indicated by a dotted line in FIG. 12, by employing n for the approximation original pixel value that is n\hat{ }2 (n is a natural number) having the smallest difference with respect to the original pixel value corresponding to each pixel square root as a value after the fraction processing of each pixel square root, an increase in quantization noise can be suppressed.

The above-described “fraction processing of employing n for the approximation original pixel value that is n\hat{ }2 (n is a natural number) having the smallest difference with respect to the original pixel value corresponding to each pixel square root as a value after the fraction processing of each pixel square root” can be specifically performed on the basis of the following Expressions (2) and (3). The meaning of each symbol of the following Expressions (2) and (3) is similar to that of the above Expression (1).

$$n = IntegerPart [ Sqrt [ x ] ] \qquad Expression ( 2 )$$

$$A = IntegerPart [ 1 + n - Sqrt [ 1 / 2 + n + {n}^{⋀} 2 ] + Sqrt [ x ] ] \qquad Expression ( 3 )$$

Then, the word length of the second compressed image data d12 is reduced by the word length adjustment unit 23 (see FIG. 8) of the image processing device 12, and the third compressed image data d13 is acquired (S4 in FIG. 10).

The extent of the data word length reduced by the word length adjustment unit 23 is not limited, but it is preferable that the word length of each pixel value of the second compressed image data d12 is reduced according to the magnitudes (particularly, the maximum values) of the plurality of pixel values included in the second compressed image data d12. That is, the word length adjustment unit 23 can reduce the word length of each pixel value of the second compressed image data d12 to the number of digits that can indicate all the pixel values included in the second compressed image data d12 (that is, the number of digits that can indicate the maximum value of the pixel value).

For example, in a case where the pixel value d1 indicating the maximum value in the image data do has the number of significant digits of 16 bits, the pixel value has the number of significant digits of 8 bits by the above-described square root calculation and integer conversion. Thus, the word length adjustment unit 23 may reduce the word length of each pixel value of the second compressed image data d12 and acquire the third compressed image data d13 in which each pixel value has a word length of 8 bits.

As described above, the word length adjustment unit 23 can cast (reduce) the word length of the pixel data to half. By thus halving the word length of the pixel data, the data amount can be halved as described above, and the influence of shot noise can be reduced to about 1 [LSB] (see FIG. 11 described above).

Then, additional compression processing of the third compressed image data d13 is performed by the compression processing unit 24 (see FIG. 8) of the image processing device 12, and the fourth compressed image data d14 is acquired (S5 in FIG. 10).

Then, the fourth compressed image data d14 is stored in the storage unit 13 as the compressed image data d50 by the storage processing unit 25 (see FIG. 8) of the image processing device 12 (S6 in FIG. 10).

The additional compression processing performed by the compression processing unit 24 is not limited, and may be lossless compression processing or lossy compression such as JPEG.

As described above, the additional compression processing is performed on the image data in which the shot noise is reduced and the data amount is compressed by the above-described processing steps S2 to S4, whereby the compression rate of the image data is further increased.

Note that, in a case where the word length of each pixel data has become half or less by the processing in the above-described processing steps S2 to S4, the image data amount has also become half or less, and thus it can be said that a sufficient image compression rate has already been achieved. Therefore, the image data (compressed image data) acquired as a result of processing steps S2 to S4 described above may be stored in the storage unit 13 without performing this processing step S5.

However, the noise component (shot noise) reduced through the above-described processing steps S2 to S4 is information having no meaning for the observer, and is an element that increases the information entropy that defines the limit of the image compression rate.

Thus, in a case where the additional image compression processing is performed on the image data in which the noise component is sufficiently reduced by the above-described processing steps S2 to S4, a better result can be expected in terms of the processing speed and the compression rate in the additional image compression processing.

As described above, in general, compression processing using DCT compression such as JPEG may result in loss of details of a dark portion of an image. However, by performing the above-described processing steps S2 to S4 as pre-stage compression processing prior to the DCT compression, loss of details of the dark portion of the image due to the subsequent DCT compression (additional image compression processing) can be reduced.

In particular, while the dynamic range of each pixel data is effectively compressed by the square root calculation processing (S2 in FIG. 10), reduction in the dynamic range of the image after decompression is suppressed, so that details of the dark portion of the image tend not to be lost even if additional image compression processing is performed.

Furthermore, the amount of image data subjected to such additional compression processing is half or less of the original amount of image data by the compression processing in the preceding processing steps S2 to S4. Thus, the data transfer time and the compression processing operation amount required for the additional compression processing are reduced, and the overall processing speed of the additional compression processing is increased.

In addition, the image data amount after the additional compression processing is compressed to half or less (about 1/10 at times) of the original image data amount. Thus, the data transfer time when the compressed image data d50 (fourth compressed image data d14) is stored in the storage unit 13 is significantly shortened, the storage capacity required for the storage unit 13 can be significantly reduced, and the storage cost can be reduced.

In addition, in the image data (pixel data), in order to ensure a wide dynamic range in the conventional method, it is necessary to secure a word length of a corresponding length. For example, when the word length of the image data (pixel data) is about 16 bits, a general-purpose compression library that can be used for the additional compression processing is limited. On the other hand, since the word length of the image data is sufficiently reduced by the above-described processing steps S2 to S4, the range of selection of the general-purpose compression library that can be used for the additional compression processing is expanded.

### [Image Decompression Processing]

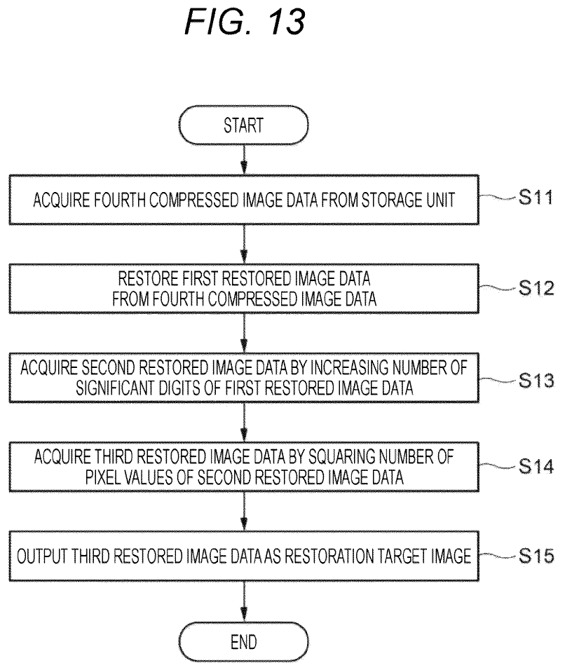

FIG. 13 is a flowchart illustrating an example of image decompression processing.

The image decompression processing can be executed on the basis of a procedure reverse to the above-described image compression processing.

That is, first, the compressed image data d50 (fourth compressed image data d14 in the present embodiment) is acquired from the storage unit 13 by the image processing device 12 (for example, the decompression processing unit 31) (S11 in FIG. 13).

Then, decompression processing on the compressed image data d50 is performed by the decompression processing unit 31, and the first decompressed image data d21 is acquired (S12). The decompression processing here is processing corresponding to the compression processing (see S5 in FIG. 10) by the compression processing unit 24 described above.

Then, the word length of the first decompressed image data d21 is increased by the word length restoration unit 32, and the second decompressed image data d22 is acquired (S13). The word length increasing processing here is processing corresponding to the word length reducing processing (see S4 in FIG. 10) by the word length adjustment unit 23 described above.

Then, by the decoding unit 33, each of the plurality of pixel values included in the second decompressed image data d22 is squared to obtain a plurality of squared pixel values, and the plurality of squared pixel values is multiplied by the reference pixel value to obtain a plurality of decompressed pixel values (S14). The square processing here is processing corresponding to the square root calculation processing (see S2 in FIG. 10) by the square root calculation unit 21 described above.

Note that, in this processing step S14, the decoding unit 33 may alternatively obtain a plurality of decompressed pixel values by squaring a value obtained by multiplying each of a plurality of pixel values included in the second decompressed image data d22 by a square root of the reference pixel value.

Then, the output processing unit 34 outputs the third decompressed image data d23 including the plurality of decompressed pixel values obtained through the series of processing steps S11 to S14 to the display device 14 as the decompressed image data d100 (S15). As a result, the decompressed image data d100 is displayed on the display device 14.

Note that, intuitively, there may be a concern that the quantization noise increases by the square root calculation processing (see S2 in FIG. 10) performed in the above-described image compression processing as compared with a case where the square root calculation processing is not performed.

However, in the above-described image compression processing, in principle, shot noise is included in the pixel data at a ratio equivalent to the quantization level, and thus, actually, an increase in quantization noise is quite small.

The inventor of the present application has compared original pixel data including shot noise (original image data) with decompressed pixel data (decompressed image data) obtained from the original pixel data through the above-described image compression processing and image decompression processing.

As a result, the inventor of the present application confirmed that no obvious noise increase was observed in the decompressed pixel data, and that the magnitude of noise included in the decompressed pixel data was suppressed to 15% or less of the magnitude of noise included in the original pixel data for almost all pixel value intensities.

As described above, according to the present embodiment, it is possible to advantageously perform the image compression processing of the image data do while reducing the noise of the image data do.

That is, the reduction of shot noise and the reduction of the amount of stored image data can be achieved using the property that the shot noise is equal to or proportional to the square root of the pixel signal value (luminance value) and the property that the number of significant digits of the pixel signal value is halved by calculation of the square root of the pixel signal value.

In this way, by mathematically adjusting the image data so that the quantization error is equivalent to the shot noise that cannot be suppressed in principle, the inevitable shot noise component is removed from the image data, and the image data amount is reduced.

Therefore, for any pixel, data compression is performed so that the minimum value (1 [LSB]) of each pixel becomes the shot noise floor, and a signal component meaningful as the subject image is basically maintained. Thus, in any pixel, data amount compression in a state in which the dynamic range is substantially maintained is achieved. For example, in a case where the image data is a biological tissue image, it is possible to suppress the amount of stored data to half or less of the original amount of image data while ensuring a wide dynamic range and maintaining the texture unique to the tissue image.

As described above, the image compression processing and the image decompression processing of the present embodiment do not or hardly sacrifice intrinsic information in image data.

In addition, since the operation in the image compression processing and the image decompression processing of the present embodiment is relatively simple, and the operation of each pixel data is a single operand operation, high-speed processing can be performed, and parallel processing by a graphics processing unit (GPU) or the like can be easily achieved.

As described above, the above-described image compression technology and image decompression technology according to the present embodiment are based on a very simple method, but have physical and mathematical precise rationality, and are also excellent in versatility.

In addition, since the image data amount and the information entropy can be reduced, the efficiency of the additional compression processing is also improved.

Furthermore, since the data amount of the image data stored in the storage unit 13 is greatly reduced, the storage cost can be reduced. In addition, since the data transfer time is shortened and the storage processing time can be greatly shortened, stagnation of other processing performed before and after the storage processing can be suppressed, and improvement in processing efficiency of the entire system and shortening of the processing time can also be expected.

In particular, a wide-area image such as a multispectral image or slide imaging tends to have an enormous amount of data, but according to the present embodiment, it is possible to perform high-speed image storage processing and image decompression processing of an image having such an enormous amount of data.

### Application Example

The device and the method of the above-described embodiments are merely examples, and an object to which the above-described image compression technology and image decompression technology can be applied is not limited.

### [Microscope System]

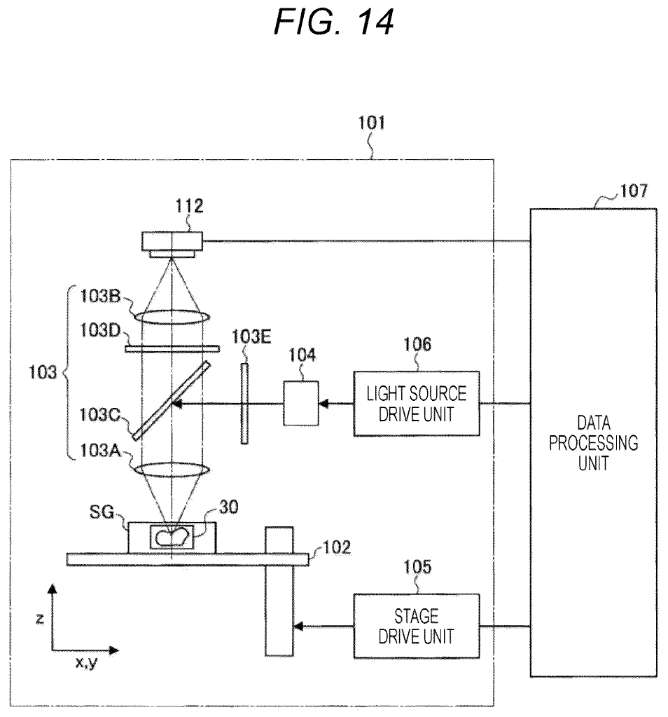

FIG. 14 is a block diagram illustrating a configuration example of a microscope system.

The microscope system illustrated in FIG. 14 includes a microscope 101 and a data processing unit 107. FIG. 14 illustrates an example of a measurement system capable of capturing an image of a wide visual field region of fluorescent-stained specimen 30 and a fluorescent-unstained specimen, and the measurement system is also applicable to, for example, Whole Slide Imaging (WSI).

The microscope 101 includes a stage 102, an optical system 103, a light source 104, a stage drive unit 105, a light source drive unit 106, and a fluorescence signal acquisition unit 112.

The stage 102 has a placement surface on which the fluorescent-stained specimen 30 and the fluorescent-unstained specimen can be placed, and is provided to be movable in a horizontal direction (an x-y plane direction) parallel to the placement surface and a vertical direction (a z-axis direction), by driving of the stage drive unit 105. The fluorescent-stained specimen 30 has a thickness of, for example, several μm to several tens μm in the Z-axis direction, and is fixed by a predetermined technique while being sandwiched between a slide glass SG and a cover glass (not illustrated).

The optical system 103 is disposed above the stage 102. The optical system 103 includes an objective lens 103A, an imaging lens 103B, a dichroic mirror 103C, an emission filter 103D, and an excitation filter 103E. The light source 104 is, for example, a light bulb such as a mercury lamp, a light emitting diode (LED), or the like, and emits light by driving of the light source drive unit 106. The light emitted from the light source 104 is guided to the fluorescent-stained specimen 30 or the fluorescent-unstained specimen on the placement surface of the stage 102, via the optical system 103.

In a case of obtaining fluorescence images of the fluorescent-stained specimen 30 and the fluorescent-unstained specimen, the excitation filter 103E generates excitation light by transmitting only light having an excitation wavelength for exciting the fluorescent dye among light emitted from the light source 104. The dichroic mirror 103C reflects the excitation light transmitted through the excitation filter 103E and incident thereon, and guides the excitation light to the objective lens 103A. The objective lens 103A condenses the excitation light on the fluorescent-stained specimen 30. The objective lens 103A and the imaging lens 103B magnify the image of the fluorescent-stained specimen 30 to a predetermined magnification, and form the magnified image on the imaging surface of the fluorescence signal acquisition unit 112.

When the fluorescent-stained specimen 30 is irradiated with excitation light, a stain (fluorescent reagent) and an autofluorescence component bound to each tissue of the fluorescent-stained specimen 30 emit fluorescence. This fluorescence is transmitted through the dichroic mirror 103C via the objective lens 103A, and reaches the imaging lens 103B via the emission filter 103D. The emission filter 103D absorbs a part of the light enlarged by the objective lens 103A and transmitted through the excitation filter 103E, and transmits only a part of color light. As described above, an image of the color light in which the external light is lost is enlarged by the imaging lens 103B, and formed on the fluorescence signal acquisition unit 112.

Note that a spectroscope (not illustrated) may be provided instead of the imaging lens 103B illustrated in FIG. 14. The spectroscope can be configured using one or more prisms, lenses, and the like, and disperses fluorescence from the fluorescent-stained specimen 30 or the fluorescent-unstained specimen in a predetermined direction. In this case, the fluorescence signal acquisition unit 112 is configured as a photodetector that detects light intensity for each wavelength of fluorescence dispersed by the spectroscope, and inputs a detected fluorescence signal to the data processing unit 107.

The data processing unit 107 drives the light source 104 via the light source drive unit 106, acquires fluorescence spectra/fluorescence images of the fluorescent-stained specimen 30 and the fluorescent-unstained specimen by using the fluorescence signal acquisition unit 112, and performs various types of processing by using the acquired fluorescence spectra/fluorescence images.

As described above, in the microscope system illustrated in FIG. 14, at least the light source 104, the excitation filter 103E, the dichroic mirror 103C, and the objective lens 103A serve as a light irradiation unit that emits excitation light for exciting the fluorescent reagent. Furthermore, the fluorescence signal acquisition unit 112 serves as an imaging device that images a specimen (the fluorescent-stained specimen 30 or the fluorescent-unstained specimen) being irradiated with excitation light to acquire a specimen fluorescence spectrum. Furthermore, the data processing unit 107 serves as an information processing device that analyzes the specimen fluorescence spectrum.

Note that the above-described device described with reference to FIG. 14 is merely an example, and the measurement system according to the above-described embodiment and modifications is not limited to the example illustrated in FIG. 14. For example, the microscope system may not necessarily include all of the configurations illustrated in FIG. 14, or may include a configuration not illustrated in FIG. 14.

The above-described embodiment and modifications can be implemented using a measurement system capable of acquiring image data (hereinafter referred to as “wide visual field image data”) with a sufficient resolution for the entire image-capturing target region or a necessary region (hereinafter also referred to as “region of interest”) in the image-capturing target region. For example, the above-described embodiments and modifications can be implemented using a measurement system capable of capturing an image of the entire image-capturing target region or a necessary region of the image-capturing target region at one time, or a measurement system that acquires an image of the entire image-capturing region or the region of interest by line scanning.

In the microscope system illustrated in FIG. 14, in a case where the entire image-capturing region is WSI exceeding a region where image data can be acquired in one time of image-capturing (hereinafter referred to as “visual field”), image-capturing of each visual field is sequentially performed by moving the stage 102 for each time of image-capturing. By tiling image data (hereinafter referred to as “visual field image data”) obtained by image-capturing of each visual field, wide visual field image data of the entire image-capturing region is generated.

The imaging processing system 10 illustrated in FIG. 7 described above may be applied to the microscope system illustrated in FIG. 14. That is, in the microscope system illustrated in FIG. 14, the “fluorescence signal acquisition unit 112” may be used as the imaging element 11 illustrated in FIG. 7, and the “data processing unit 107” may be used as the image processing device 12, the storage unit 13, the display device 14, and the imaging processing control unit 15 illustrated in FIG. 7. Alternatively, other devices in addition to the “data processing unit 107” or in place of the “data processing unit 107” may be used as the image processing device 12, the storage unit 13, the display device 14, and the imaging processing control unit 15 illustrated in FIG. 7.

### [Imaging Device]

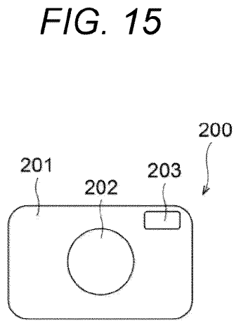

FIG. 15 is a diagram illustrating an example of the imaging device 200.

An imaging device 200 illustrated in FIG. 15 includes an imaging optical system 202 and an imaging light emitting unit 203 mounted on an imaging main body unit 201.

The imaging device 200 is configured as what is called a digital camera, and can be configured as a compact camera with no interchangeable lens or an interchangeable lens camera (for example, a single-lens reflex camera or a mirrorless camera). However, the specific configuration and application of the imaging device 200 are not limited.

The imaging processing system 10 illustrated in FIG. 7 described above may be applied to the imaging device 200 illustrated in FIG. 15. That is, the imaging element 11, the image processing device 12, the storage unit 13, the display device 14, and the imaging processing control unit 15 illustrated in FIG. 7 may be mounted on the imaging main body unit 201 illustrated in FIG. 15.

### [Mobile Device]

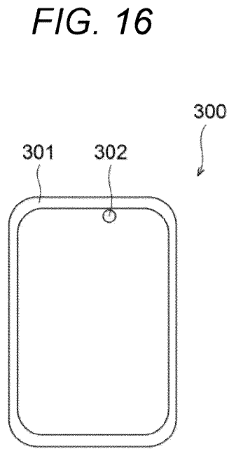

FIG. 16 is a diagram illustrating an example of the mobile device 300.

A mobile device 300 illustrated in FIG. 16 includes a device imaging unit 302 mounted on a device main body unit 301. The device imaging unit 302 illustrated in FIG. 16 is configured as what is called a front camera, but the mobile device 300 may include a rear camera (not illustrated) mounted on the back side of the device main body unit 301.

The mobile device 300 is typically configurable as a mobile phone, smartphone or other tablet terminal. However, a specific configuration and application of the mobile device 300 are not limited.

The imaging processing system 10 illustrated in FIG. 7 described above may be applied to the mobile device 300 illustrated in FIG. 16. That is, the imaging element 11, the image processing device 12, the storage unit 13, the display device 14, and the imaging processing control unit 15 illustrated in FIG. 7 may be mounted on the device main body unit 301 illustrated in FIG. 16.

### [Client Server System]

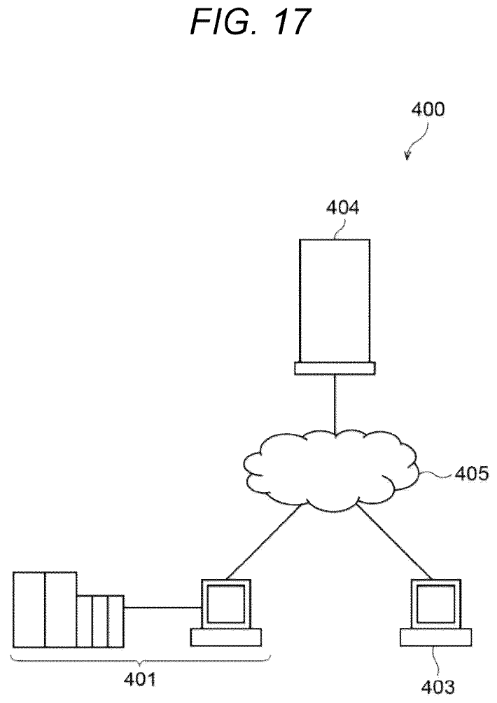

FIG. 17 is a diagram illustrating an example of the client server system 400.

A client server system 400 illustrated in FIG. 17 includes a client 401, an information processing device 403, and a server 404 connected to each other via a network 405.

The client 401 and the information processing device 403 can upload and store various data to the server 404 via the network 405, and can download and acquire various data stored in the server 404 and/or calculated by the server 404.

In response to a request (command signal) from the client 401 and the information processing device 403, the server 404 can store various kinds of data transmitted from the client 401 and the information processing device 403, transmit various kinds of stored data, or perform various kinds of data processing.

For example, the image data do may be transmitted from the client 401 and/or the information processing device 403 to the server 404 via the network 405. In this case, the server 404 may function as the image processing device 12 and the storage unit 13 described above. That is, the server 404 may acquire the compressed image data d50 by performing the above-described image compression processing (see FIGS. 8 and 10) on the image data do transmitted from the information processing device 403, and store the compressed image data d50 in a storage unit (not illustrated) included in the server itself. Furthermore, the server 404 may acquire the decompressed image data d100 by performing the above-described image decompression processing (see FIGS. 9 and 13) on the compressed image data d50, and transmit the decompressed image data d100 to the client 401 and/or the information processing device 403.

Note that the server 404 may perform only one of the above-described image compression processing (see FIGS. 8 and 10) and image decompression processing (see FIGS. 9 and 13).

For example, the server 404 may perform the above-described image compression processing (see FIGS. 8 and 10) on the image data do transmitted from the client 401 and/or the information processing device 403 via the network 405, and store the compressed image data d50. Then, the server 404 may transmit the compressed image data d50 to the client 401 and/or the information processing device 403 in response to the request without performing the image decompression processing. In this case, the client 401 and/or the information processing device 403 functions as the above-described image processing device 12, and performs the above-described image decompression processing (see FIGS. 9 and 13) on the received compressed image data d50 to acquire the decompressed image data d100.

Furthermore, the server 404 may store the compressed image data d50 transmitted from the client 401 and/or the information processing device 403 via the network 405, perform image decompression processing on the compressed image data d50 in response to a request, and transmit the decompressed image data d100. In this case, the client 401 and/or the information processing device 403 functions as the above-described image processing device 12, and performs the above-described image compression processing (see FIGS. 8 and 10) on the image data do to acquire the compressed image data d50. On the other hand, the server 404 also functions as the above-described image processing device 12, and performs the above-described image decompression processing (see FIGS. 9 and 13) on the compressed image data d50 to acquire the decompressed image data d100.

Furthermore, the server 404 need not function as the above-described image processing device 12, and may function only as the above-described storage unit 13. That is, the server 404 may store the compressed image data d50 transmitted from the client 401 and/or the information processing device 403 via the network 405, and transmit the compressed image data d50 to the client 401 and/or the information processing device 403 in response to a request. In this case, the client 401 and/or the information processing device 403 function as the above-described image processing device 12.

Note that the client 401 and the information processing device 403 may have functions equivalent to each other or may play roles different from each other. For example, the information processing device 403 may exclusively transmit the image data do or the compressed image data d50 to the server 404, while the client 401 may exclusively acquire the compressed image data d50 or the decompressed image data d100 from the server 404.

It should be noted that the embodiments and modifications disclosed in the present description are illustrative only in all respects and are not to be construed as limiting. The above-described embodiments and modifications can be omitted, replaced, and changed in various forms without departing from the scope and spirit of the appended claims. For example, the above-described embodiments and modifications may be combined in whole or in part, and other embodiments may be combined with the above-described embodiments or modifications. Furthermore, the effects of the present disclosure described in the present description are merely exemplification, and other effects may be provided.

A technical category embodying the above technical idea is not limited. For example, the above-described technical idea may be embodied by a computer program for causing a computer to execute one or a plurality of procedures (steps) included in a method of manufacturing or using the above-described device. Furthermore, the above-described technical idea may be embodied by a computer-readable non-transitory recording medium in which such a computer program is recorded.

### Supplementary Note

The present disclosure can also have the following configurations.

### Item 1

An image processing device including:
a square root calculation unit that acquires first compressed image data by calculating a plurality of pixel square roots that are square roots of a plurality of original pixel values based on a plurality of pixel values included in image data; and
a fraction processing unit that acquires second compressed image data by performing fraction processing of the plurality of pixel square roots.

### Item 2

The image processing device according to item 1, in which
the plurality of original pixel values is derived by dividing a plurality of pixel values included in the image data by a reference pixel value that is an output value corresponding to one electron in photoelectric conversion processing of the imaging element that has acquired the image data.

### Item 3

The image processing device according to item 1 or 2, in which
the fraction processing unit acquires the second compressed image data by rounding a numerical value after a decimal point of each of the plurality of pixel square roots on a basis of rounding off.

### Item 4

The image processing device according to any one of items 1 to 3, in which
the fraction processing unit obtains an approximation original pixel value that is n\hat{ }2 (n is a natural number) having a smallest difference with respect to an original pixel value corresponding to each of the pixel square roots for each of the plurality of pixel square roots, and employs n of the approximation original pixel value as a pixel value of the second compressed image data.

### Item 5

The image processing device according to any one of items 1 to 4, further including:
a word length adjustment unit that acquires third compressed image data by reducing a word length of the second compressed image data according to sizes of a plurality of pixel values included in the second compressed image data.

### Item 6

The image processing device according to item 5, in which
the word length adjustment unit acquires the third compressed image data by reducing the word length of the second compressed image data according to a number of significant digits of a maximum value among the plurality of pixel values included in the second compressed image data.

### Item 7

The image processing device according to item 5 or 6, in which
the second compressed image data has a word length equal to or more than 16 bits, and
the third compressed image data has a word length equal to or less than 8 bits.

### Item 8

The image processing device according to any one of items 1 to 7, further including:
a compression processing unit that acquires fourth compressed image data by performing compression processing on compressed image data based on the second compressed image data.

### Item 9

The image processing device according to item 8, further including:
a word length adjustment unit that acquires third compressed image data by reducing a word length of the second compressed image data according to sizes of a plurality of pixel values included in the second compressed image data, in which
the compression processing unit acquires fourth compressed image data by performing compression processing on the third compressed image data.

### Item 10

The image processing device according to any one of items 1 to 9, further including:
a storage processing unit that causes compressed image data (for example, the second compressed image data, the third compressed image data, or the fourth compressed image data) based on the second compressed image data to be stored in a storage unit.

### Item 11

The image processing device according to any one of items 1 to 10, further including:
a decoding unit that performs processing of squaring a plurality of pixel values included in compressed image data (for example, the second compressed image data, the third compressed image data, or the fourth compressed image data) based on the second compressed image data.

### Item 12

The image processing device according to item 11, further including:
a word length restoration unit that increases a word length of the compressed image data based on the second compressed image data, in which
the decoding unit performs processing of squaring a plurality of pixel values included in the compressed image data based on the second compressed image data after the word length of the compressed image data based on the second compressed image data is increased by the word length restoration unit.

### Item 13

The image processing device according to item 11 or 12, further including:
a compression processing unit that acquires fourth compressed image data by performing compression processing of the compressed image data based on the second compressed image data; and
a decompression processing unit that performs decompression processing on the compressed image data based on the second compressed image data, in which
the compressed image data to be decompressed is based on the fourth compressed image data, and
the decompression processing is processing corresponding to the compression processing performed by the compression processing unit.

### Item 14

The image processing device according to any one of items 11 to 13, in which
the plurality of original pixel values is derived by dividing a plurality of pixel values included in the image data by a reference pixel value that is an output value corresponding to one electron in photoelectric conversion processing of the imaging element that has acquired the image data, and
the decoding unit acquires a plurality of decompressed pixel values by multiplying a plurality of squared pixel values obtained by squaring a plurality of pixel values included in the compressed image data based on the second compressed image data by the reference pixel value.

### Item 15

The image processing device according to any one of items 1 to 14, in which
the image data is biological tissue image data.

### Item 16

The image processing device according to item 15, in which
the biological tissue image data is fluorescent antibody image data.

### Item 17

An image processing method including:
a step of acquiring first compressed image data by calculating a plurality of pixel square roots that are square roots of a plurality of original pixel values based on a plurality of pixel values included in image data; and
a step of acquiring second compressed image data by performing fraction processing of the plurality of pixel square roots.

### Item 18

A program for causing a computer to execute:
a procedure of acquiring first compressed image data by calculating a plurality of pixel square roots that are square roots of a plurality of original pixel values based on a plurality of pixel values included in image data; and
a procedure of acquiring second compressed image data by performing fraction processing of the plurality of pixel square roots.

### REFERENCE SIGNS LIST

10 Imaging processing system
11 Imaging element
12 Image processing device
13 Storage unit
14 Display device
15 Imaging processing control unit
21 Square root calculation unit
22 Fraction processing unit
23 Word length adjustment unit
24 Compression processing unit
25 Storage processing unit
31 Decompression processing unit
32 Word length restoration unit
33 Decoding unit
34 Output processing unit
80 Photon
81 Pixel
82 Electron
83 Electronic storage unit
101 Microscope
102 Stage
103 Optical system
103A Objective lens
103B Imaging lens
103C Dichroic mirror
103D Emission filter
103E Excitation filter
104 Light source
105 Stage drive unit
106 Light source drive unit
107 Data processing unit
112 Fluorescence signal acquisition unit
200 Imaging device
201 Imaging main body unit
202 Imaging optical system
203 Imaging light emitting unit
300 Mobile device
301 Device main body unit
302 Device imaging unit
400 Client server system
401 Client
403 Information processing device
404 Server
405 Network
d0 Image data
d1 Pixel value
d11 First compressed image data
d12 Second compressed image data
d13 Third compressed image data
d14 Fourth compressed image data
d21 First decompressed image data
d22 Second decompressed image data
d23 Third decompressed image data
d50 Compressed image data
d100 Decompressed image data

1. An image processing device comprising:
a square root calculation unit that acquires first compressed image data by calculating a plurality of pixel square roots that are square roots of a plurality of original pixel values based on a plurality of pixel values included in image data; and
a fraction processing unit that acquires second compressed image data by performing fraction processing of the plurality of pixel square roots.

2. The image processing device according to claim 1, wherein
the plurality of original pixel values is derived by dividing a plurality of pixel values included in the image data by a reference pixel value that is an output value corresponding to one electron in photoelectric conversion processing of the imaging element that has acquired the image data.

3. The image processing device according to claim 1, wherein
the fraction processing unit acquires the second compressed image data by rounding a numerical value after a decimal point of each of the plurality of pixel square roots on a basis of rounding off.

4. The image processing device according to claim 1, wherein
the fraction processing unit obtains an approximation original pixel value that is n\hat{ }2 (n is a natural number) having a smallest difference with respect to an original pixel value corresponding to each of the pixel square roots for each of the plurality of pixel square roots, and employs n of the approximation original pixel value as a pixel value of the second compressed image data.

5. The image processing device according to claim 1, further comprising:
a word length adjustment unit that acquires third compressed image data by reducing a word length of the second compressed image data according to sizes of a plurality of pixel values included in the second compressed image data.

6. The image processing device according to claim 5, wherein
the word length adjustment unit acquires the third compressed image data by reducing the word length of the second compressed image data according to a number of significant digits of a maximum value among the plurality of pixel values included in the second compressed image data.

7. The image processing device according to claim 5, wherein
the second compressed image data has a word length equal to or more than 16 bits, and
the third compressed image data has a word length equal to or less than 8 bits.

8. The image processing device according to claim 1, further comprising:
a compression processing unit that acquires fourth compressed image data by performing compression processing on compressed image data based on the second compressed image data.

9. The image processing device according to claim 8, further comprising:
a word length adjustment unit that acquires third compressed image data by reducing a word length of the second compressed image data according to sizes of a plurality of pixel values included in the second compressed image data, wherein
the compression processing unit acquires fourth compressed image data by performing compression processing on the third compressed image data.

10. The image processing device according to claim 1, further comprising:
a storage processing unit that causes compressed image data based on the second compressed image data to be stored in a storage unit.

11. The image processing device according to claim 1, further comprising:
a decoding unit that performs processing of squaring a plurality of pixel values included in compressed image data based on the second compressed image data.

12. The image processing device according to claim 11, further comprising:
a word length restoration unit that increases a word length of the compressed image data based on the second compressed image data, wherein
the decoding unit performs processing of squaring a plurality of pixel values included in the compressed image data based on the second compressed image data after the word length of the compressed image data based on the second compressed image data is increased by the word length restoration unit.

13. The image processing device according to claim 11, further comprising:
a compression processing unit that acquires fourth compressed image data by performing compression processing of the compressed image data based on the second compressed image data; and
a decompression processing unit that performs decompression processing on the compressed image data based on the second compressed image data, wherein
the compressed image data to be decompressed is based on the fourth compressed image data, and
the decompression processing is processing corresponding to the compression processing performed by the compression processing unit.

14. The image processing device according to claim 11, wherein
the plurality of original pixel values is derived by dividing a plurality of pixel values included in the image data by a reference pixel value that is an output value corresponding to one electron in photoelectric conversion processing of the imaging element that has acquired the image data, and
the decoding unit acquires a plurality of decompressed pixel values by multiplying a plurality of squared pixel values obtained by squaring a plurality of pixel values included in the compressed image data based on the second compressed image data by the reference pixel value.

15. The image processing device according to claim 1, wherein
the image data is biological tissue image data.

16. The image processing device according to claim 15, wherein
the biological tissue image data is fluorescent antibody image data.

17. An image processing method comprising:
a step of acquiring first compressed image data by calculating a plurality of pixel square roots that are square roots of a plurality of original pixel values based on a plurality of pixel values included in image data; and
a step of acquiring second compressed image data by performing fraction processing of the plurality of pixel square roots.

18. A program for causing a computer to execute:
a procedure of acquiring first compressed image data by calculating a plurality of pixel square roots that are square roots of a plurality of original pixel values based on a plurality of pixel values included in image data; and
a procedure of acquiring second compressed image data by performing fraction processing of the plurality of pixel square roots.
